(19) United States
(12) Patent Application Publication
(10) Pub. No.: US 2012/0121064 A1
(43) Pub. Date: May 17, 2012

(54) PROCEDURE FOR PROCESSING PATIENT RADIOLOGICAL IMAGES

(76) Inventor: Sylvain Bernard, Montigny le Bretonneux (FR)

(21) Appl. No.: 13/284,029

(22) Filed: Oct. 28, 2011

(30) Foreign Application Priority Data
Nov. 16, 2010 (FR) 1059410

(51) Int. Cl. A61B 6/00; G06K 9/00

(52) U.S. Cl. 378/37; 382/132

(57) ABSTRACT

A medical imaging process using an imaging system is provided. The process includes: acquiring a first plurality of 2D projection images of the object via x-ray emission along a plurality of orientations selected with respect to the object's craniocaudal direction, one of the orientations being the object's craniocaudal direction; obtaining a reconstructed 3D volume of the object along the craniocaudal direction from the first plurality of 2D projection images acquired; obtaining a reconstructed 2D craniocaudal image; acquiring a second plurality of 2D projection images of the object via x-ray emission along a plurality of orientations selected with respect to the object's mediolateral-oblique direction, one of the orientations being the object's mediolateral-oblique direction; obtaining a reconstructed 3D volume of the object along the mediolateral-oblique direction from the second plurality of 2D projection images acquired; and obtaining a reconstructed 2D mediolateral-oblique image.

### BACKGROUND OF THE INVENTION

1. Field of the Invention

The present disclosure relates to medical imagery processes and devices such as mammography and tomosynthesis in the context of detection or diagnosis of breast disease or tumours.

2. Description of the Prior Art

Detection or diagnostic operations for identifying tumours in subjects' breasts are carried out by mammography, tomosynthesis or the two in combination.

Mammography makes it possible to obtain 2D images of an object of interest, the subject's breast in the present case.

For its part, tomosynthesis makes it possible to obtain 3D modelling of a subject's object of interest, which is achieved by 3D reconstruction of the object of interest from a plurality of 2D projections of this zone along distinct directions. The 3D modelling is typically carried out by thick or thin cuts, corresponding respectively to slices having relatively large thickness and zero-thickness slices of the object of interest being examined.

Due to the recent development of this technique, however, it is frequently paired with mammography so that users have 2D images available, with which they are accustomed to work, in order to facilitate interpretation and comparison with images made earlier.

In the case of mammography and tomosynthesis, two exposures are often used to make 2D or 3D images. Craniocaudal (CC) exposure, which consists of irradiating the breast from above, in a direction running substantially from the subject's head to her feet, allowing 2D CC images or 3D CC modelling to be obtained, and mediolateral-oblique (MLO) exposure, which consists of irradiating the breast in an oblique direction, allowing 2D MLO images or 3D MLO modelling to be obtained.

Carrying out craniocaudal (CC) exposures requires the x-ray source to move very near the subject's head. In order to reduce subject discomfort and to limit subject's exposure to x-ray emissions, solutions have been proposed. In particular, in document FR 2 882 246 by the applicant, a protective screen for the subject's head is disclosed. In document FR 2 881 338 by the applicant, an asymmetric acquisition with respect to the CC axis is disclosed.

In the case of screening operations which consist of carrying out detection within a population of subjects, it is desired to limit the x-ray dose to which subjects are exposed. The number of exposures is therefore limited, so as not to expose the population to an excessive dose of radiation. A dose is the quantity of x-rays emitted in order to carry out an exposure, whether the exposure is 3D CC, 3D MLO, 2D CC or 2D MLO.

Thus, conventional arrangements typically propose making a 2D image along either of the CC or the MLO direction, and 3D modelling along the other of the MLO or CC direction; a 2D CC image associated with 3D MLO modelling, for example.

To carry out these exposures, x-rays are emitted toward the subject's breast. Conventional methods then use a quantity of x-ray emissions corresponding to two doses to produce: one 2D CC image and one 2D MLO image (mammography); one 3D CC image and one 3D MLO image (tomosynthesis); one 3D CC image and one 2D MLO image (mixed); or one 2D CC image and one 3D MLO image (mixed).

The absence of a 3D image is penalizing in terms of accuracy and level of detail; the absence of a 2D image is penalizing for the practitioner in terms of ease of interpretation and comparison with data recorded earlier, and the mixed solutions do not allow complete results to be obtained in either the CC or the MLO direction.

Solutions have been suggested that implement four separate acquisition steps, one for each of the following views: 2D CC, 2D MLO, 3D CC and 3D MLO. A greater x-ray emission dose results from these four acquisition steps, each exposure requiring one dose for a total of four doses, or more generally, one dose of x-rays emitted corresponding to the equivalent of four conventional 2D exposures, which is not satisfactory in the context of screening operations.

### BRIEF SUMMARY OF THE INVENTION

Embodiments of the present invention provide an imaging process that does not have these disadvantages.

According to an embodiment of the present invention, a medical imaging process using an imaging system comprising an x-ray beam source set facing a detector upon which an object is placed is provided. The process comprises: acquiring a first plurality of 2D projection images of the object via x-ray emission along a plurality of orientations selected with respect to the object's craniocaudal direction, one of the orientations being the object's craniocaudal direction; obtaining a reconstructed 3D volume of the object along the craniocaudal direction from the first plurality of 2D projection images acquired; obtaining a reconstructed 2D craniocaudal image; acquiring a second plurality of 2D projection images of the object via x-ray emission along a plurality of orientations selected with respect to the object's mediolateral-oblique direction, one of the orientations being the object's mediolateral-oblique direction; obtaining a reconstructed 3D volume of the object along the mediolateral-oblique direction from the second plurality of 2D projection images acquired; and obtaining a reconstructed 2D mediolateral-oblique image.

According to another embodiment of the present invention, a medical imaging system for viewing an object is provided. The medical imagining system comprises: a base extending in a plane; an arm, movable with respect to the base; a beam source carried on the movable arm; a beam detector configured to detect a beam emitted by the beam source; and a processing unit. The processing unit may be configured to control the movement of the arm and the emission of x-rays by the beam source in the subject's craniocaudal and mediolateral-oblique directions; determine reconstruction slices of the object along the subject's craniocaudal and mediolateral-oblique directions and a reconstructed 3D volume of the object in the subject's craniocaudal direction and a reconstructed 3D volume of the object of interest in the subject's mediolateral-oblique direction; obtain a reconstructed 2D craniocaudal image from reconstruction slices and projections in the subject's craniocaudal direction; and obtain a reconstructed 2D mediolateral-oblique image of the object from reconstruction slices and projections in the subject's mediolateral-oblique direction.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 shows a schematic representation of a medical imaging system according to embodiments of the present invention;

FIG. 2 illustrates a process of reconstructing 2D images from the 3D modelling according to an embodiment of the present invention;

FIGS. 3, 4, 5 and 6 show variations of a process of reconstructing 2D images from the 3D modelling in according to embodiments of the present invention; and

FIG. 7 shows an imaging process according to embodiments of the present invention.

### DETAILED DESCRIPTION OF THE INVENTION

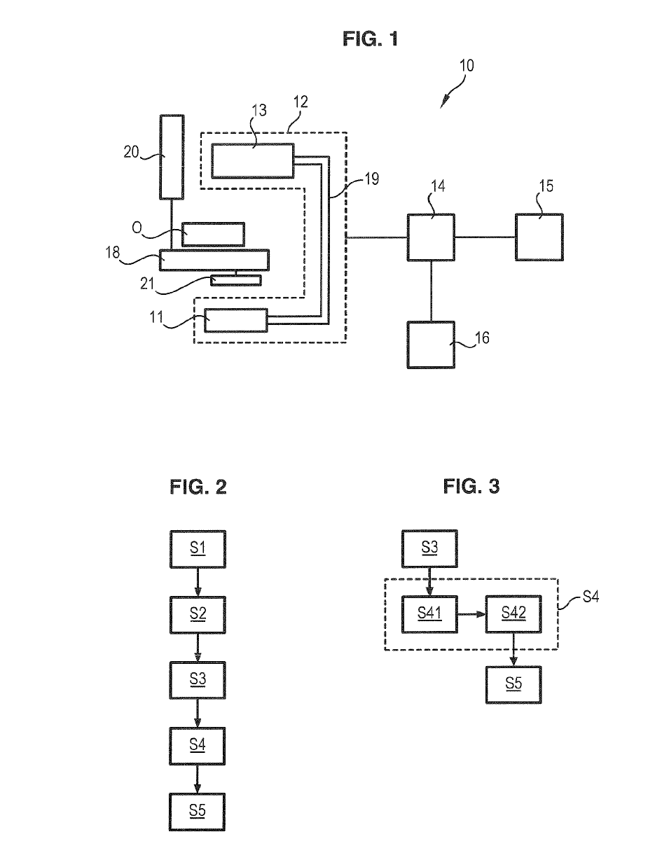

FIG. 1 shows a schematic representation of a medical imaging system 10 according to embodiments of the present invention.

The medical imaging system as illustrated includes an acquisition unit 12, an image processing unit 14, a display unit 16 and a base 18 designed so that a subject's object of interest O can be placed there.

The image acquisition unit 12 allows the acquisition of a plurality of 2D projections of the object of interest O, typically an organ or a breast of a subject. It includes in particular a detector 11 located facing a beam source 13, detector 11 and source 13 being typically located at two separate ends of an arm 19, movable with respect to base 18.

The display unit 16 may be integrated into image acquisition unit 12 or image processing unit 14, or may be separate from it. The display unit may be, for instance, a screen.

The display unit 16 makes it possible, in particular, for the practitioner to view the exposures carried out by the medical imaging system 10.

Processing unit 14 is designed for implementing processing procedures, for example for implementing 3D reconstruction processes making 3D modelling possible based on a plurality of 2D projections, or for obtaining reconstructed 2D images, which will be described hereinafter.

The processing unit may include, for example, one or more computers, one or more processors, microcontrollers, etc.

Processing unit 14 is coupled to a memory unit 15, which can be integral with or separate from processing unit 14. This memory unit allows storage of data such as 2D images or 3D modelling, and may be, for example, a hard disk, a CD-ROM, a diskette, ROM/RAM memory or any other suitable means.

Processing unit 14 may include a reader (not shown), for example, a diskette reader or a CD-ROM reader, to read image processing instructions from an instruction medium (not shown), such as a diskette or a CD-ROM. As a variation, processing unit 14 executes image processing instructions stored in microcode (not shown).

As a variation, imaging system 10 can include a screen 20 protecting the subject's head, designed to be held in a fixed position with respect to the subject while exposures are made, between the trajectory of the beam source 12 and the subject's head so as to protect the subject's head from the beam emitted by source 12.

Further, imaging system 10 can also be provided with an anti-diffusion grid 21, comprising a plurality of opaque components arranged parallel to one another, in a direction parallel to the motion of the movable arm.

Such anti-diffusion grids are in fact required in mammography, to limit the impact of the spread of emitted x-rays within the subject's body.

As an example, document FR 2 939 019 by the applicant discloses anti-diffusion grids.

Embodiments of the present invention are based on a specific image processing procedure allowing a 2D image similar to a mammography image to be obtained from images obtained by tomosynthesis, called a reconstructed 2D image. The image visually resembles a standard 2D full dose mammography image obtained by emission of a dose of x-rays.

Reconstructed 2D images allow a practitioner to easily make out the specific features of the object of interest, and to make comparisons with older images carried out by standard mammography. They also make it possible to present the practitioner with an image format with which he is accustomed to working, unlike the 3D modellings of tomosynthesis, which are relatively recent.

The image processing procedure therefore consists of processing radiographic images obtained by an imaging system 10 including an emission source 13 arranged facing a detector 11 on which the object of interest O is placed.

FIG. 2 illustrates a procedure for processing images obtained by tomosynthesis so as to obtain reconstructed 2D images.

In a first step S1, a plurality of 2D projection images of the object of interest O is obtained using a plurality of orientations, one so-called zero orientation being that closest to the reference direction selected, to which each orientation is referred.

During this first step, one 2D image in particular is acquired at a selected orientation. In one embodiment, the selected orientation corresponds to the selected reference direction.

The process then typically includes a step S2 of applying a filter to the acquired 2D projection images so as to obtain filtered projection images of the object of interest O.

This filter may be of the high-pass type and its cut-off frequency may be determined according to the thickness of the object of interest O.

During step S3, reconstruction slices of the object of interest O are determined. This step S3 consists in particular of back-projection of the filtered 2D projection images obtained in step S2.

This back-projection may in particular be of the nonlinear, “Order Statistics Based Backprojection” type. In linear back-projection, each voxel of the volume is reconstructed using N pixels of information, each pixel being determined by projection of the voxel into each of the N projections. In nonlinear back-projection, the maximum intensity pixel among the N is not used, which makes it possible to considerably reduce the replication artefacts caused by the most intense objects.

It is noted that the reconstruction slices of the object of interest O represent the reconstructed volume of the object of interest O. This step therefore consists essentially of obtaining the reconstructed 3D volume of the object of interest (O) along the selected orientation direction, typically the craniocaudal direction or the mediolateral-oblique direction.

Thereafter, during step S4, a re-projection of the reconstruction slices is carried out in the selected reference direction. This makes it possible to obtain an intermediate 2D image of the object of interest O. It is noted that re-projection is done in the same direction as the projection image corresponding to the selected reference direction.

Finally, in step S5, a reconstructed 2D image is obtained of the object of interest by combining the intermediate 2D image and the projection image corresponding to the selected reference direction. The combination may be a linear, pixel to pixel combination.

The reconstructed 2D image is similar to a mammography image.

In one embodiment, the re-projection step S4 of re-projecting reconstruction slices 50 is an MIP (“Maximum Intensity Pixel”) re-projection in the selected orientation direction. More generally, any re-projection involving sorting pixel values present along the radii can be used (SIP, for “Sorted Intensity Pixel”). The sort consists of classifying pixels by their intensity (ascending or descending sort).

FIG. 3 illustrates the steps in MIP re-projection according to an embodiment of the present invention. The re-projection step S4 is then made up of two sub-steps S41 and S42, described below.

This type of MIP re-projection consists of a determination S41, for each pixel of an image, typically the intermediate 2D image, within the volume consisting of filtered fine slices, of the maximum-intensity voxel along the radius extending from the source to the pixel, and a step S42 involving storing, in memory unit 14 of the imaging system, an identifier for the reconstruction slice in which the maximum-intensity voxel is located.

In this manner, depth information is available, in memory unit 14, connecting each pixel in the intermediate 2D image with the associated reconstruction slice from which the pixel is derived.

As a variation, the re-projection of the thin slices is an SIP re-projection in the selected orientation direction, the SIP re-projection consisting of a determination, for each pixel of the intermediate 2D image, within the volume consisting of filtered thin slices, of a voxel whose intensity is calculated using a sort of the voxel values along the radius extending from the source to the pixel.

Re-projection can be implemented differently from the way presented above.

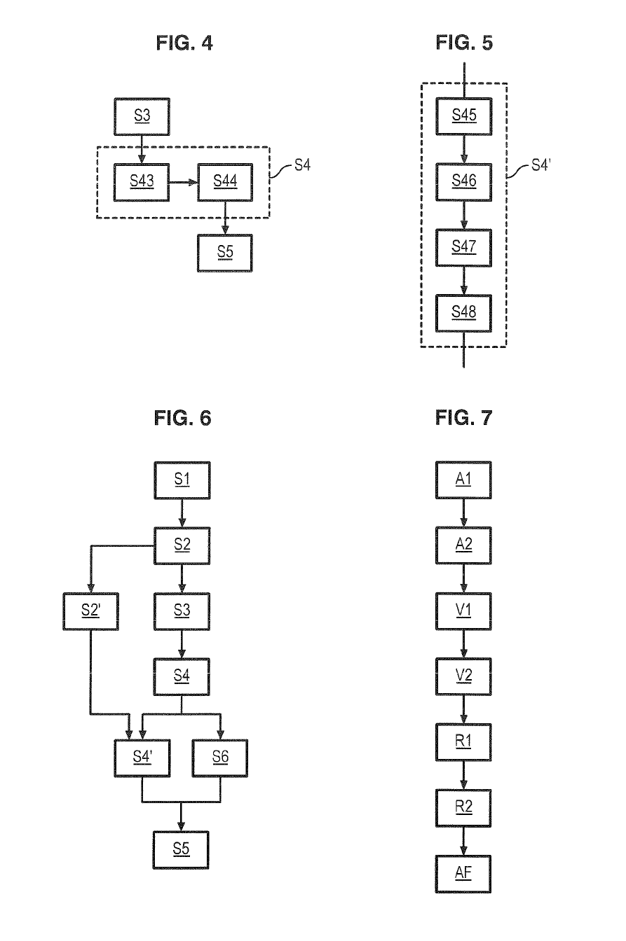

FIG. 4 illustrates re-projection steps according to an alternate embodiment of the present invention.

In this embodiment, the re-projection S4 of the reconstruction slices consists additionally of any pixel of the intermediate 2D image in a selection S43 of the voxel having the highest probability of belonging to a lesion along the radius extending from the source to that pixel and a step S44 involving storage in memory in memory unit 14 of the imaging system, of an identifier for the reconstruction slice in which the maximum-probability voxel is located.

This assumes that each voxel has a probability of belonging to a lesion associated with it. An automatic detection system (3D CAD, for “Computer-Aided Detection”) makes it possible to obtain such a volume of probabilities.

In this fashion, depth information is placed in memory unit 14, connecting each pixel in the intermediate 2D image to the associated reconstruction slice from which the pixel is derived.

This processing procedure may also include a local step S6 in which processing unit 13 smoothes the depth information. This smoothing is typically carried out following step S4, and consists of making the information more locally uniform. The result is depth information.

The process may include a step S2′ in which the processing unit 13 applies a filter to the projection image along the reference direction of the object of interest O, before step S5 in which the final 2D image is prepared, so as to reduce the noise in that final 2D image. The filter applied is may be a low-pass filter. This step S2′ accomplishes a filtering of the 2D projection images acquired during the craniocaudal A1 and mediolateral-oblique A2 acquisition steps.

Further, it is possible to implement a step S4′ in which display slices are determined that correspond to the reconstructed volume of the object of interest. In other words, it is a volume obtained from the projection images by reconstruction processes known in the state of the art whose objective is the visualization of slices. The process then includes a step S45 in which thin slices of the 3D volume of the object of interest O are obtained in the craniocaudal CC direction and the mediolateral-oblique MLO direction.

The reconstructed 3D volume is typically a volume made up of thick slices, which is advantageous for example for the detection of lesions, because it makes it possible particularly to quickly view them in their entirety.

In addition, a reconstruction in thick slices is advantageous in terms of the volume of data, which is considerably smaller than the corresponding set of thin slices.

According to an embodiment of the present invention, the thick slices are of uniform thickness, and each of the thick slices overlaps the adjacent thick slices halfway. In another embodiment, thin slices of the object of interest O are also displayed.

According to an embodiment of the present invention, obtaining a thick slice is accomplished via the following steps: a step S46 involving filtering the thin slices making up the thick slice, typically with a high-pass filter; a step S47 of re-projection of the fine slices at the mean height of the thick slice; and a step S48 in which the re-projection image of the thin slices is combined with the filtered image of the thin slice at the mean height of the thick slice.

FIG. 5 illustrates a subdivision of the determination step S4′ and sub-steps S45, S46, S47 and S48.

FIG. 6 illustrates the process as described previously, in which steps S6, S2′ and S4′ are integrated.

The process as illustrated in FIG. 6 includes step S2′, carried out following step S2, the result of which is used during step S4′. This step S4′ is carried out following step S4, typically in parallel with step S6, prior to step S5.

An embodiment of the present invention includes carrying out two iterations of the process: one iteration for making a reconstructed 3D volume and a reconstructed 2D image in the craniocaudal (CC) direction, and one iteration for making a reconstructed 3D volume and a reconstructed 2D image in the mediolateral-oblique (MLO) direction, from the projections in the craniocaudal (CC) and in the mediolateral-oblique (MLO) direction, respectively.

The process according to an embodiment of the present invention includes: two tomosynthesis image acquisition steps A1 and A2, in the craniocaudal and mediolateral-oblique directions respectively; two steps V1 and V2 for obtaining reconstructed 3D volumes for each of these directions; and two steps R1 and R2 for obtaining reconstructed 2D images for each of these directions.

The order in which the steps are carried out may vary. In one embodiment, the two acquisition steps may be carried out, then, once the acquisitions are carried out, the steps of obtaining the reconstructed volumes and 2D images may be carried out.

The steps may also be carried out sequentially according to direction, and thus a first acquisition step in a first direction (CC or MLO) followed by the steps for obtaining a reconstructed 3D volume and a reconstructed 2D image for this direction, then a second acquisition step in a second direction (MLO or CC) followed by the steps for obtaining a reconstructed 3D volume and a reconstructed 2D image for that direction may be carried out.

The process according to one embodiment of the present invention may include an additional step AF for displaying the reconstructed 3D modellings and 2D images thus carried out.

According to an embodiment of the present invention, this display is carried out by displaying: on a first screen or part of a screen, the reconstructed 3D volume of the object of interest O in the craniocaudal direction and alternatively the corresponding reconstructed 2D image; and on a second screen or part of a screen, the reconstructed 3D volume of the object of interest O in the mediolateral-oblique direction and alternatively the corresponding reconstructed 2D image.

FIG. 7 illustrates a schematic representation of the process according to an embodiment of the present invention, and illustrates the case where the two acquisition steps A1 and A2 are carried out prior to two steps V1 and V2 for obtaining reconstructed 3D volumes, and two steps for obtaining reconstructed 2D images R1 and R2 corresponding respectively to the CC and MLO reconstructed acquisitions and 2D, or vice versa.

The process includes a display step AF, which, in one embodiment, may be broken down into two sub-steps of: a first sub-step of displaying 3D CC modelling and 3D MLO modelling only; and a second sub-step of displaying reconstructed 2D CC and reconstructed 2D MLO images. The second sub-step may occur only after the user has viewed the corresponding 3D modellings, typically once the user has viewed all the thin slices and/or thick slices constituting the 3D view.

Presenting the display of the reconstructed 2D images this way makes it possible to ensure that the practitioner will take notice of information contained in the 3D modelling, and will not limit himself to the reconstructed 2D images which, by superimposing tissues, can hide lesions. They can in fact allow easier interpretation by the practitioner, but the information is to be extracted from the 3D modellings.

In one embodiment of the present invention, the process can include an additional step in which a user modifies the reconstructed 2D images, so as to point out in them regions of interest.

For example, a user can identify in the 3D modelling specific areas or volumes of interest in the subject's breast and note their position on the reconstructed 2D images. This identification can also be carried out automatically, typically by means of a calculator.

This marking on the reconstructed 2D images can be carried out directly by a user on the reconstructed 2D images, or a user can point out these zones on the 3D modellings, and a processing unit will then transfer them automatically to the reconstructed 2D images via re-projection of these identified volumes of interest.

The process may include a step for displaying, by user action, thin or thick slices of the object of interest that intersect the identified volume of interest.

Acquisition steps A1 and A2 may include various features that are detailed hereafter.

In one embodiment of the present invention, the orientations along which the 2D projections of the object of interest are carried out in the acquisition step in the craniocaudal direction are symmetrical with respect to the craniocaudal direction. In fact, conventionally, the orientations are distributed on both sides of the craniocaudal direction, symmetrically for example. Craniocaudal acquisition can, however, cause discomfort for the subject, in that the beam source 13 or detector 11 are near the head.

In order to correct this disadvantage, the CC direction acquisition step can be carried out with 2D projections of the object of interest along orientations distributed non-symmetrically with respect to the craniocaudal direction.

Such an asymmetric distribution of orientations along which the 2D projections of the object of interest are carried out make it possible to dispense with the use of a protective screen for the subject's head as described earlier.

According to an embodiment of the present invention, the x-ray dose sent can be distributed uniformly or not along the various orientations.

As an example, if for the acquisition step in either the CC or the MLO direction, five orientations are selected, taking as a reference unit one dose of x-rays, a uniform distribution of this dose would lead to a quantity of x-rays emitted equal to ⅕ along each of the five orientations.

It can, however, be advantageous to distribute this dose in a non-uniform manner, typically by emitting a greater quantity of x-rays for the orientations closest to the selected reference direction (CC or MLO) and a smaller quantity of the more distant orientations.

The invention thus makes it possible to obtain a complete set of data on an object of interest, in the form of 3D modelling (3D CC and 3D MLO), which also presents two corresponding reconstructed 2D images (reconstructed 2D CC and reconstructed 2D MLO) so as to facilitate the practitioner's interpretation.

The invention makes it possible to obtain this complete and easily interpreted data set without requiring a greater x-ray dose than conventional processes limited to two 2D images, or one 2D image and a 3D image.

To this end, the invention makes use of a process for reconstructing a 2D image from a plurality of 2D projections along a plurality of orientations, instead of resorting to an additional acquisition step which would increase the x-ray dose injected into the subject.

In addition, in the case where the imaging device used in implementing the process includes an anti-diffusion grid, the invention then makes it possible to obtain 3D modellings in the presence of an anti-diffusion grid.

What is claimed is:

1. A medical imaging process using an imaging system comprising an x-ray beam source set facing a detector upon which an object is placed, the process comprising:
acquiring a first plurality of 2D projection images of the object via x-ray emission along a plurality of orientations selected with respect to the object's craniocaudal direction, one of the orientations being the object's craniocaudal direction;
obtaining a reconstructed 3D volume of the object along the craniocaudal direction from the first plurality of 2D projection images acquired;
obtaining a reconstructed 2D craniocaudal image;
acquiring a second plurality of 2D projection images of the object via x-ray emission along a plurality of orientations selected with respect to the object's mediolateral-oblique direction, one of the orientations being the object's mediolateral-oblique direction;
obtaining a reconstructed 3D volume of the object along the mediolateral-oblique direction from the second plurality of 2D projection images acquired; and
obtaining a reconstructed 2D mediolateral-oblique image.

2. The medical imaging process according to claim 1, wherein obtaining a reconstructed 3D volume of the object along the craniocaudal direction from the first plurality of 2D projection images acquired comprises obtaining thin slices of the object along the craniocaudal direction.

3. The medical imaging process according to claim 1, wherein obtaining a reconstructed 3D volume of the object along the mediolateral-oblique direction from the second plurality of 2D projection images acquired comprises obtaining thin slices of the object along the mediolateral-oblique direction.

4. The medical imaging process according to claim 1, wherein the reconstructed 3D volumes of the object along the craniocaudal direction and along the mediolateral-oblique direction are thick slices.

5. The medical imaging process according to claim 1, wherein the reconstructed 3D volumes of the object along the craniocaudal direction and along the mediolateral-oblique direction are thick, fixed thickness slices, wherein each thick slice half-overlaps the adjacent thick slices.

6. The medical imaging process according to claim 5, wherein obtaining a reconstructed 3D volume of the object along the craniocaudal direction from the first plurality of 2D projection images acquired and obtaining a reconstructed 3D volume of the object along the mediolateral-oblique direction from the second plurality of 2D projection images acquired further comprise:
filtering thin slices that make up the thick slices;
re-projecting the thin slices at the mean height of the thick slice; and
combining the re-projection images with the filtered images at the mean height of the thick slice.

7. The medical imaging process according to claim 6, wherein filtering the thin slices is performed with a high-pass filter.

8. The medical imaging process according to claim 6, wherein re-projecting the thin slices comprises an SIP re-projection in the selected orientation direction, the SIP re-projection comprising determining a voxel whose intensity is calculated using a sort of the voxel values along the radius extending from the source to the pixel of the mean height slice, wherein a voxel is determined for each pixel of the re-projection image.

9. The medical imaging process according to claim 6, wherein re-projecting the thin slices comprises an MIP re-projection in the selected orientation direction, the MIP re-projection comprising determining the maximum intensity voxel along the radius extending from the source to the mean height pixel wherein the maximum intensity voxel is determined for each pixel of the re-projection image within the volume consisting of thin filtered slices.

10. The medical imaging process according to claim 1, wherein the plurality of orientations selected with respect to the object's craniocaudal direction are distributed asymmetrically with respect to the craniocaudal direction of the object.

11. The medical imaging process according to claim 1, wherein obtaining a reconstructed 2D craniocaudal image comprises;
filtering the first plurality of 2D projection images acquired;
determining reconstruction slices of the object from the plurality of filtered 2D images;
re-projecting the reconstruction slices along the subject's craniocaudal direction so as to obtain an intermediate 2D craniocaudal image; and
obtaining a reconstructed 2D craniocaudal image of the object by combining the intermediate 2D craniocaudal image and the projection image corresponding to the craniocaudal direction, and
wherein obtaining a reconstructed 2D mediolateral-oblique image comprises:
filtering the second plurality of 2D projection images acquired;
determining reconstruction slices of the object from the plurality of filtered 2D projection images;
re-projecting the reconstruction slices along the subject's mediolateral-oblique direction so as to obtain an intermediate 2D mediolateral-oblique image; and
obtaining a reconstructed 2D mediolateral-oblique image of the object by combining the intermediate 2D mediolateral-oblique image and the projection image corresponding to the mediolateral-oblique direction.

12. The medical imaging process according to claim 1 further comprises displaying at least one of:
the reconstructed 3D volume of the object of interest in the craniocaudal directionl
the reconstructed 2D image in the craniocaudal direction;
the reconstructed 3D volume of the object of interest in the mediolateral-oblique direction; and
the reconstructed 2D image in the mediolateral-oblique direction.

13. The medical imaging process according to claim 12, wherein the imaging system further comprises a display unit, and wherein displaying comprises:
displaying on a first screen or on at least part of a screen, the reconstructed 3D volume of the object in the craniocaudal direction, and alternatively the corresponding reconstructed 2D image; and
displaying on a second screen or at least part of a screen, the reconstructed 3D volume of the object in the mediolateral-oblique direction, and alternatively the corresponding reconstructed 2D image.

14. The medical imaging process of claim 1, further comprising storing volumes of interest in memory, and re-projecting the volumes of interest onto the corresponding reconstructed 2D image.

15. The medical imaging process according to claim 14, further comprising selecting a volume of interest re-projected onto at least one of the reconstructed 2D craniocaudal image and the reconstructed 2D mediolateral-oblique image, and further comprising displaying thin or thick slices of the object that intersect the selected volume of interest.

16. A medical imaging system for viewing an object, the medical imagining system comprising:
a base extending in a plane;
an arm, movable with respect to the base;
a beam source carried on the movable arm;
a beam detector configured to detect a beam emitted by the beam source; and
a processing unit configured to:
control the movement of the arm and the emission of x-rays by the beam source in the subject's craniocaudal and mediolateral-oblique directions;
determine reconstruction slices of the object along the subject's craniocaudal and mediolateral-oblique directions and a reconstructed 3D volume of the object in the subject's craniocaudal direction and a reconstructed 3D volume of the object of interest in the subject's mediolateral-oblique direction;
obtain a reconstructed 2D craniocaudal image from reconstruction slices and projections in the subject's craniocaudal direction; and
obtain a reconstructed 2D mediolateral-oblique image of the object from reconstruction slices and projections in the subject's mediolateral-oblique direction.
